(19) United States
(12) Patent Application Publication
(10) Pub. No.: US 2009/0099440 A1
(43) Pub. Date: Apr. 16, 2009

(54) REDUCTION OF RF INDUCED TISSUE HEATING USING DISCRETE WINDING PATTERNS

(76) Inventor: Ingmar Viohl, Canyon Country, CA (US)

Correspondence Address: MICHAEL BEST & FRIEDRICH LLP, 100 E WISCONSIN AVENUE, Suite 3300, MILWAUKEE, WI, 53202, US

(21) Appl. No.: 12/117,342

(22) Filed: May 8, 2008

Related U.S. Application Data
(60) Provisional application No. 60/998,478, filed on Oct. 11, 2007.
(60) Provisional application No. 60/998,477, filed on Oct. 11, 2007.

(51) Int. Cl. A61B 5/042; A61N 1/05

(52) U.S. Cl. 600/373; 607/116

(57) ABSTRACT

The present invention provides, among other things, a medical device having an elongated body and an electrically conductive coil wrapped around the elongated body and covering at least a lengthwise portion of the body. The coil includes a pattern of insulated and non-insulated portions.

### RELATED APPLICATION

This application claims the benefit of U.S. Provisional Application No. 60/998,478, filed Oct. 11, 2007, and 60/998,477, filed Oct. 11, 2007, the contents of both of which are incorporated herein by reference.

### FIELD OF THE INVENTION

The present invention relates to methods and devices for reducing or eliminating the effects of electromagnetic fields on long metallic structures as are typically found in medical devices having leads or catheters.

### BACKGROUND

Medical devices, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like, commonly employ one or more conductive surfaces, often in the form of leads and catheters that either receive or deliver voltage, current or other electromagnetic pulses from or to an organ or its surrounding tissue for diagnostic or therapeutic purposes. When exposed to electromagnetic fields, such as for example those present in magnetic resonance imaging (“MRI”) systems, these conductive surfaces may sustain undesired currents and or voltages that interact with the surrounding blood and tissue, potentially resulting in unwanted tissue heating, nerve stimulation or other negative effects resulting in erroneous diagnosis or therapy delivery.

Further, such structures commonly include bare or insulated coiled wire forming one or more tightly wound solenoid-like structures along their shafts. These tightly wound coils facilitate torque transfer, prevent “buckling” and allow the conduction of electrical signals to and from the proximal (system) end to the distal (patient) end of the device.

An example of a typical medical device incorporating conductive surfaces for the transfer of diagnostic and therapeutic electromagnetic signals as well as mechanical torque transfer is the catheter R shown in FIG. I. The catheter R includes a distal tip electrode A, which is commonly used to deliver energy to the target tissue and to receive electrical signals from the tissue it contacts. The catheter also includes three proximal electrodes B, which are typically used to receive electrical signals from the tissue they are contacting. This type of catheter structure is encountered in cardiac ablation and EP mapping catheters, for example. The electrical contact between the proximal end P of the catheter and the electrodes A and B is typically made via a bundle of individually insulated wires or conductors D. An outer coil structure C is typically used for torque transfer and is not in contact with the electrodes A and B. The outer coil C and the wires D sometimes sustain currents when exposed to an electromagnetic field, such as for example that encountered in an MRI system. These currents can, for example, induce heating or cause nerve stimulation in the tissue surrounding the device either directly or by creating current pathways through the tissue that interacts with the electrodes A and B.

A second example of a medical device incorporating conductive wires for the transfer of diagnostic and therapeutic electromagnetic signals, as well as mechanical torque transfer, is the device shown in FIG. II. This type of structure may be encountered, for example, in pacemaker or ICD leads. The lead includes a distal tip electrode A, which is commonly used to deliver energy to the target tissue and to receive electrical signals from the tissue it contacts. The lead also includes a proximal electrode B, which is mostly used to receive electrical signals from the tissue in its vicinity. In pacemaker and ICD leads, the conductive paths or coiled wires C and H are connected to the electrodes B and A, respectively, and are typically surrounded by dielectric materials E, F and G. The conductive paths provided by coiled wires C and H can sustain unwanted currents when exposed to an electromagnetic field, such as for example encountered in an MRI system. These currents can induce heating in the tissue surrounding the device either directly or by creating current pathways through the tissue involving the electrodes A and B and the pathways provided by C and H.

One approach to form the braiding of a catheter or lead, such as the structures C and H shown in FIGS. I and II, is to wind a bare, thin wire J on a flexible former I, as depicted in FIG. III. The close winding structure facilitates torque transfer between the ends of the device and prevents the device from buckling when it is pushed. The close pitched windings are in random electrical contact with each other and essentially form a continuous conductive pathway. Even though the outer structure C, the conductor or wire bundle D of FIG. I, and the inner coil structure H of FIG. II are enclosed in non-conductive tubing E, F and G, the insulating layers do not entirely prevent undesired AC currents from propagating on these structures.

In some constructions, a thin insulated wire K is used instead of the bare wire J in an attempt to form an inductor extending along the full shaft of the device, as shown in FIG. IV. The purpose of this inductor is to act as a “choke” and suppress currents from propagating along the shaft of the catheter or lead. Because of the small pitch utilized in the structure of FIG. IV, the formed coil, even with wire K insulated, may not be entirely electrically equivalent to a pure inductor over the full frequency spectrum of interest.

Other typical approaches to reduce the current and voltage induced in the catheter and lead-like structures use discrete components, often self-resonating RF chokes or LC (“tank”) circuits to block RF currents on the wires or conductors. These components literally “break” or interrupt the original conductor, which may affect the mechanical characteristics of the device and increase the potential for mechanical failure. In addition, discrete components such as capacitors are often magnetic and result in image artifacts or cannot be obtained in small enough sizes to allow the manufacture of small diameter leads and catheters.

### SUMMARY

In some embodiments, the present invention provides a medical device having one or more elongated bodies and electrically conductive coils wrapped around one or more of the elongated bodies and covering at least a lengthwise portion of one of the bodies, where the coil(s) include at least one mechanically continuous wire including at least one or more insulated sections and one or more non-insulated sections.

The present invention also provides a medical device having one or more elongated bodies and electrically conductive coils wrapped around one or more of the elongated bodies and covering at least a lengthwise portion of one of the bodies, where the coils include at least one mechanically continuous wire including at least one or more insulated sections and one or more non-insulated sections and incorporate one or more mechanically continuous non-conductive filars.

The present invention also provides a medical device having one or more elongated bodies and electrically conductive coils wrapped around one or more of the elongated bodies and covering at least a lengthwise portion of one of the bodies, where the coils include at least one mechanically continuous insulated wire and incorporate one or more mechanically continuous non-conductive filars.

The present invention also provides a medical device having one or more elongated bodies and electrically conductive coils wrapped around one or more of the elongated bodies and covering at least a lengthwise portion of one of the bodies, where the coils include at least one mechanically continuous bare wire and incorporate one or more mechanically continuous non-conductive filars.

In addition, the present invention provides a method of controlling the current induced by an electromagnetic field on a medical device including elongated conductive structures. The method includes the act of forming a string of inductors utilizing mechanically continuous wire where the inductors act as non-resonant RF chokes over a specified frequency range.

The method can also include the act of forming a string of inductors utilizing mechanically continuous wire in which one or more inductors are self-resonant RF chokes. A string including multiple inductors can incorporate self-resonant chokes at a single or multiple frequencies, as well as non-resonant RF chokes over a large frequency span.

The method can also include the act of forming multiple strings of inductors, each formed from a mechanically continuous wire, in which the strings are coaxial.

The method can also include the act of forming strings of inductors, each formed from a mechanically continuous wire, in which the strings are co-radial, i.e., form the bodies of two or more co-radial elongated conducting structures.

Other aspects of the invention will become apparent by consideration of the detailed description and accompanying drawings.

### BRIEF DESCRIPTION OF THE DRAWINGS

FIG. I is a perspective view of a typical medical device having elongated conductive pathways in the form of a wire coil and a bundle as typically found in RF ablation and EP mapping catheters.

FIG. II is a perspective view of another typical medical device incorporating an inner and outer elongated conductive pathway in the form of wire coils as typically found in pacemaker and ICD leads.

FIG. III is a perspective view of a typical conductive wire coil structure used in the devices shown in FIGS. I and II, wherein the conductive structure is formed by coiling a non-insulated wire on a cylindrical support.

FIG. IV is a perspective view of another conductive wire coil structure used in the devices shown in FIGS. I and II, wherein the conductive structure is formed by coiling an insulated wire on a cylindrical support.

FIG. 1 is a perspective view of a medical device incorporating two coaxial conductive wire coil structures according to some embodiments of the present invention.

FIG. 2 is a perspective view of another medical device incorporating two coaxial conductive wire coil structures according to some embodiments of the present invention.

FIG. 3 is a perspective view of still another medical device incorporating two coaxial conductive wire coil structures according to some embodiments of the present invention.

FIG. 4 is a perspective view of a conductive wire coil structure of FIGS. 1-3 including a string of wound inductor sections and wound non-insulated wire sections.

FIG. 4a is a magnified perspective view of a transition point of the conductive wire coil structure of FIG. 4.

FIG. 5 is a perspective view of a structurally continuous single wire used to form the conductive wire coil structure of FIG. 4.

FIG. 6 is a perspective view of a string of wound interlaced inductor sections and wound interlaced non-insulated wire sections to form another conductive wire coil structure of FIGS. 1-3.

FIG. 6a is a magnified perspective view of a transition point of the conductive wire coil structure of FIG. 6.

FIG. 7 is a perspective view of a structurally continuous set of wires used to form the multi filar conductive structure of FIG. 6.

FIG. 8 is a perspective view of a string of wound interlaced inductor sections and wound interlaced non-insulated wire sections, incorporating a discrete non-conductive turn to form still another conductive wire coil structure of FIGS. 1-3.

FIG. 9 is a perspective view of a structurally continuous set of parallel wires and a non-conductive filar used to form the multi filar wire coil structure of FIG. 8.

FIG. 10 is a perspective view of a wound set of interlaced inductor sections and a discrete non-conductive turn to form another conductive wire coil structure of FIGS. 1-3.

FIG. 11 is a perspective view of insulated wires and a non-conductive filar used to form the conductive wire coil structure of FIG. 10.

FIG. 12 is a perspective view of an inductor formed using non-insulated wire in combination with a non-conductive filar member to form yet another conductive wire coil structure of FIGS. 1-3.

FIG. 13 is a perspective view of non-insulated wires and a non-conductive filar used to form the conductive wire coil structure of FIG. 12.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, it is to be understood that phraseology and terminology used herein with reference to device or element orientation (such as, for example, terms like “central,” “upper,” “lower,” “front,” “rear,” “distal,” “proximal,” and the like) are only used to simplify description of the present invention, and do not alone indicate or imply that the device or element referred to must have a particular orientation. In addition, terms such as “first” and “second” are used herein for purposes of description and are not intended to indicate or imply relative importance or significance.

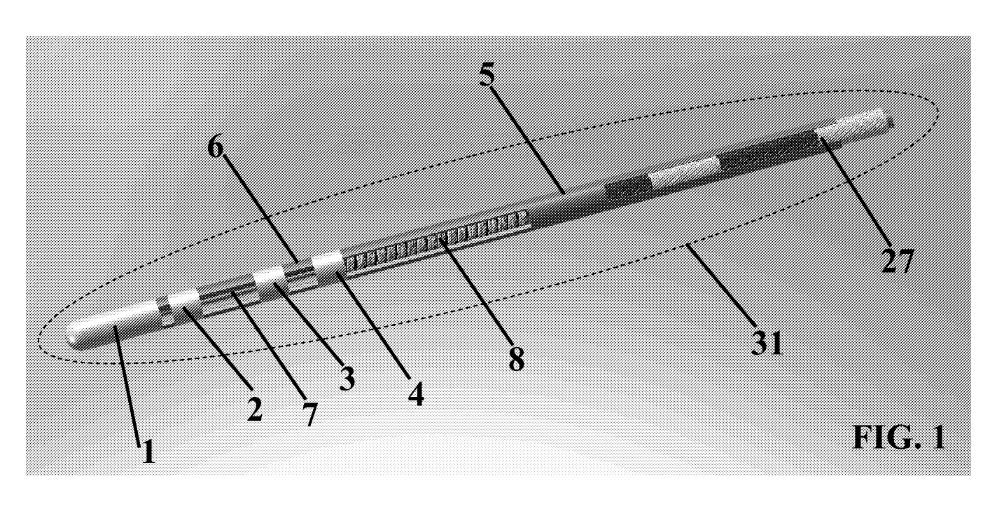

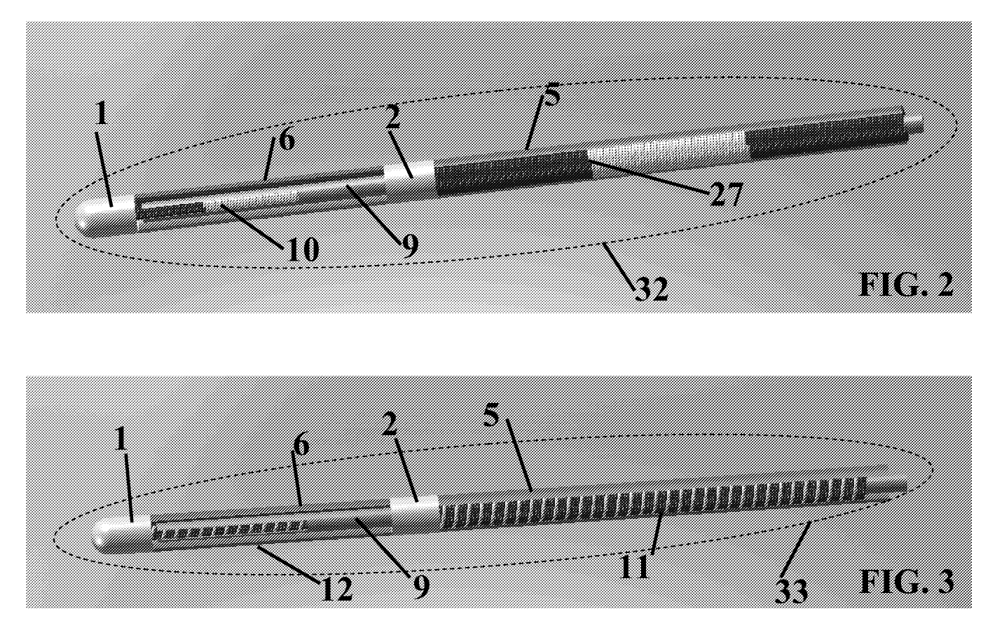

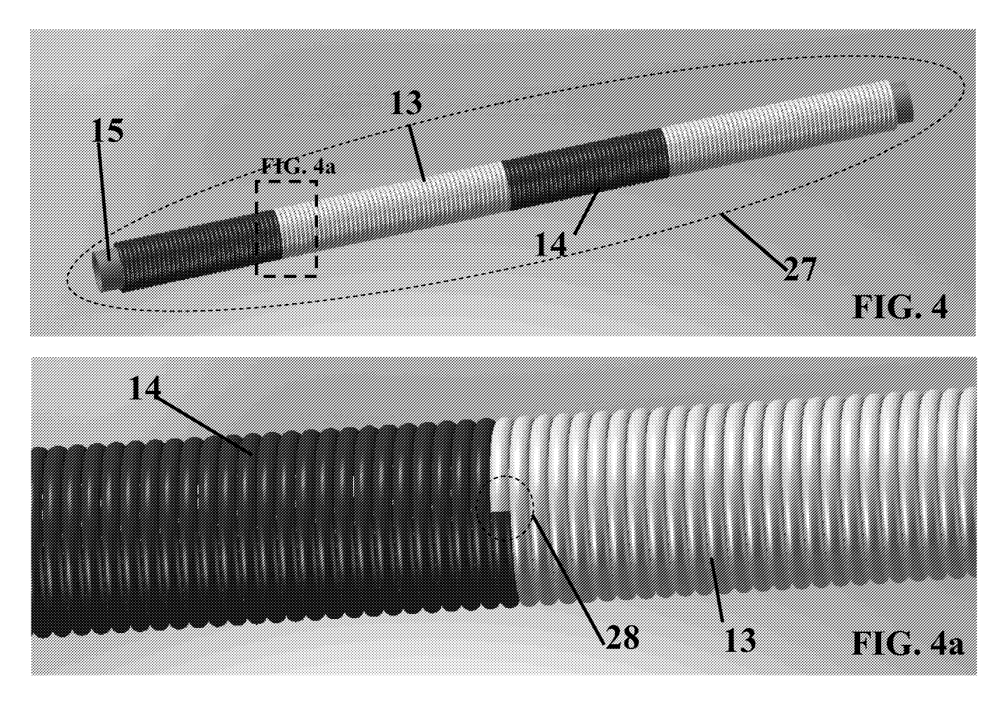

With reference to the Figures, an exemplary medical device according to the present invention is shown in FIG. 1, as a catheter 31. It will be understood by those of skill in the art that the catheter 31 could be any of a number of medical devices, including EP mapping catheters, imaging catheters, RF ablation catheters, angioplasty catheters, neurostimulator leads, etc. Second and third exemplary medical devices according to the present invention are shown in FIGS. 2 and 3. The devices 32 and 33 are shown as bipolar leads and could be any number of medical devices, including pacemaker and ICD leads. The devices shown in FIGS. 1-3 include a distal “tip” electrode 1, electrodes 2, 3, and 4, surrounding dielectric materials 5, 6, and 9, and various other structures described below. It will be further understood by those of ordinary skill in the art that the catheter and leads shown in FIGS. 1-3 could include any number of other or additional features as are commonly found in typical medical devices such as catheters and leads.

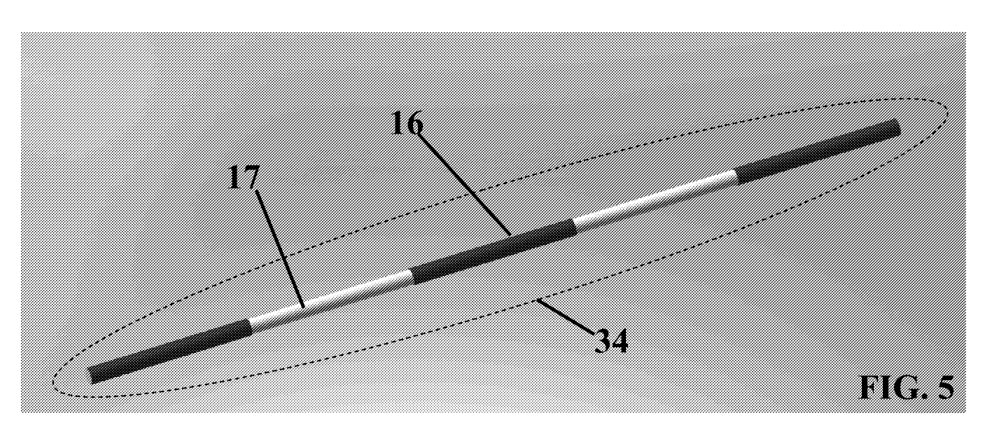

The structurally continuous, conductive wire coil structures 10 and 27 in FIGS. 1-2 electrically represent a string of one or more inductors 14 (as best seen in FIGS. 4 and 4a) and one or more bare coil sections 13 (FIGS. 4 and 4a) that, depending on the pitch, may electrically create a short circuit. The mechanically continuous wire coil structures 10 and 27 are formed by wrapping a single, continuous wire 34 (FIG. 5) around support structure 15, thus forming a conductive wire coil 27 at the proximal portion of the catheter 31 or a conductive wire coil 10 and 27 at the distal and proximal portion of lead 32. The support structure 15 is commonly used in the fabrication of catheters and leads and may consist of hollow tubing or solid rods. The base material typically is an insulating, flexible, dielectric material such as polytetrafluoroethylene (PTFE), ethylene tetrafluoroethylene (ETFE) or a silicone based polymer.

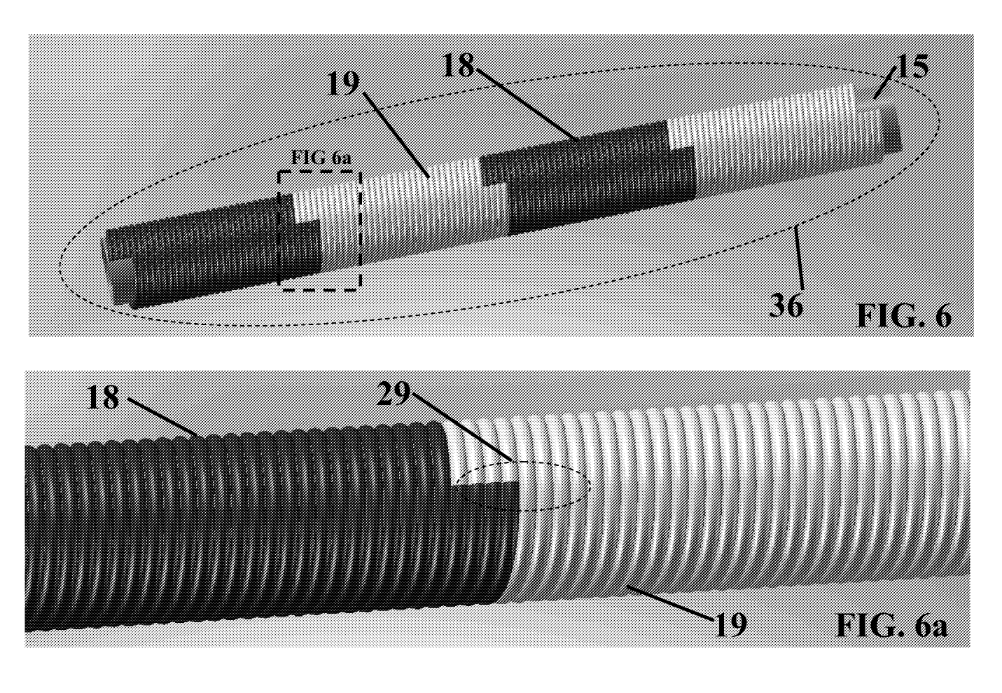

As shown in FIG. 5, the single, continuous wire 34 includes insulated sections 16 and non-insulated or bare sections 17. As a result, when the mechanically continuous wire 34 is wrapped around the support structure 15, as shown in FIG. 4, the resulting braiding or coil comprises a continuous coil having alternating insulated and non-insulated sections 14 and 13, respectively. Because the wire 34 is a mechanically continuous wire, the transition points 28 (shown in FIG. 4a) between the insulated and non-insulated sections 14 and 13 are mechanically continuous and do not require any means of joining such as soldering, welding, etc. It will be understood by those of skill in the art that instead of the single wire 34 of FIG. 5, multiple continuous wires 35 as shown in FIG. 7, could be used. Additionally, the conductive wire coil structure 27 of FIG. 4 and 36 of FIG. 6 could comprise more sections 13, 14 and 18, 19 than shown, and the size, spacing, and insulated/non-insulated pattern of sections 13, 14 and 18, 19 can be varied within the spirit and scope of the present invention.

In some embodiments, the alternating insulated and non-insulated sections 16 and 17 of the wire structure 34 are created by a removal process that removes partial sections from a fully insulated wire by chemical, mechanical, optical, or thermal means (e.g., chemical etching, mechanical grinding, laser burning, etc.). In other embodiments, the alternating insulated and non-insulated sections 16 and 17 of the wire structure 34 are created by a covering process that covers sections of a fully non-insulated (bare) wire with insulation material by means of partial extrusion, chemical deposition, etc.

In some embodiments, the alternating insulated and non-insulated sections 14 and 13 of the structures 10 and 27 are formed by initially creating the structure C of FIG. IV using fully insulated wire and subsequently removing partial sections from the fully insulated section by chemical, mechanical, optical, or thermal means. In other embodiments, the alternating insulated and non-insulated sections 14 and 13 of the structures 10 and 27 are formed by initially creating the structure C of FIG. III with bare wire and subsequently covering sections with insulation material by means of “dipping” or chemical deposition. In still other embodiments, the alternating insulated and non-insulated sections 14 and 13 are created by “joining” fully insulated and non-insulated sections by means of soldering, welding, fusing, clueing, etc.

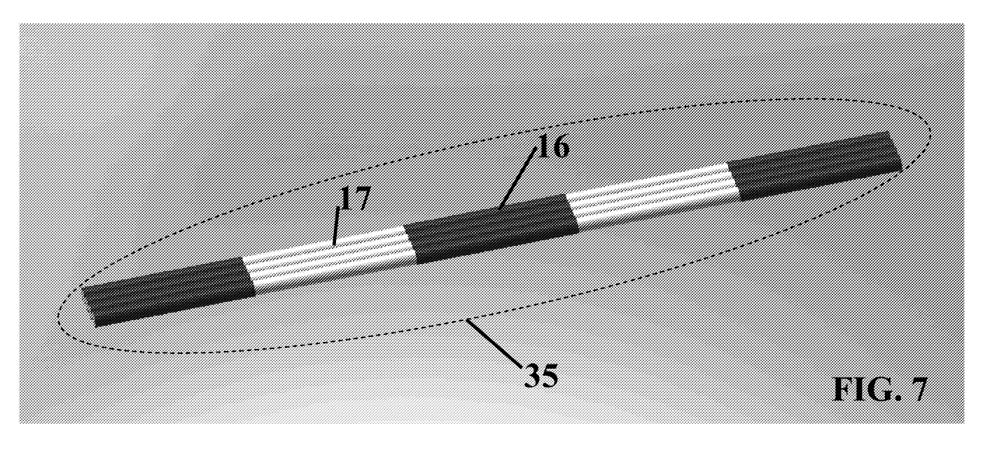

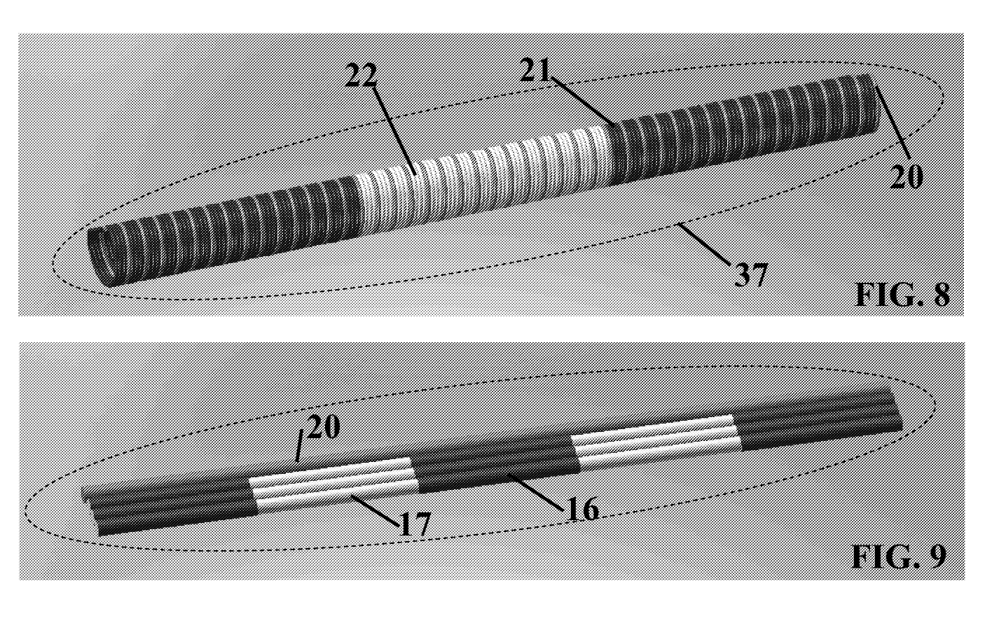

In some embodiments of the present invention, the device can include one or more braiding coils 37, 23 or 25, as shown in FIGS. 8, 10 and 12, respectively.

Braiding coil 37 includes four wires (three wires similar to wire 34 and one non-conductive member 20, for example a plastic “wire” or filament) coiled together in a quadruple helix, resulting in a pattern of conductive sections spaced by a non-conductive member 20, essentially forming a string of interlaced inductors 21 connected via an inductor 22 formed by the bare wire section. It will be understood by those of skill in the art that more or fewer wires and non-conductive members 20 can be used in varying quantities, resulting in a variety of patterns exhibiting varying electrical characteristics while maintaining similar mechanical behavior.

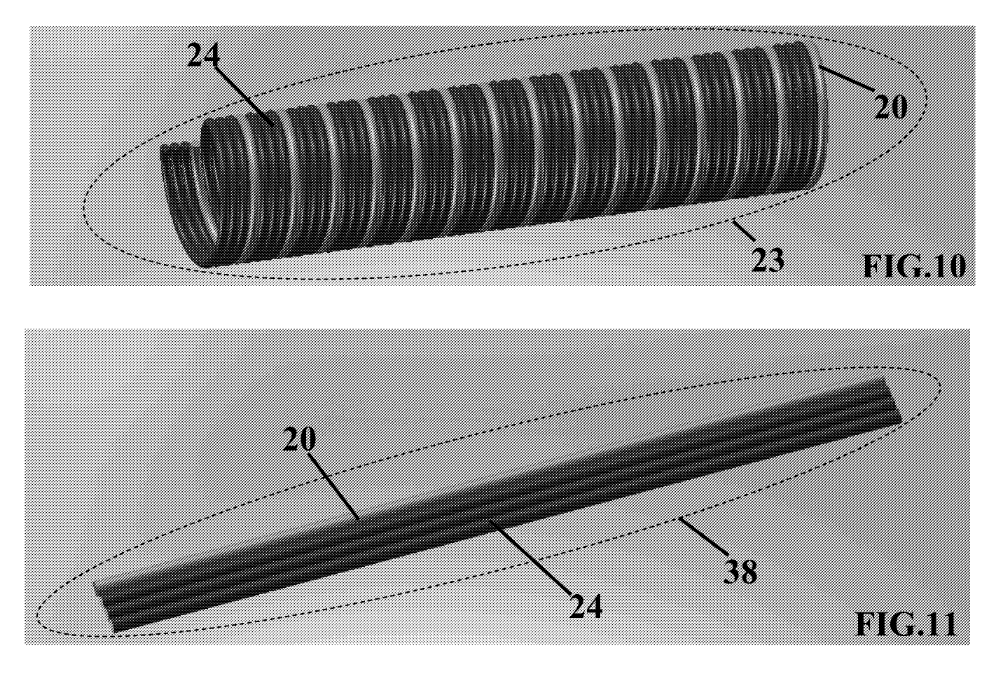

Referring to FIG. 10, braiding coil 23 includes four wires (three fully insulated wires 24 and one non-conductive member 20) coiled together in a quadruple helix, resulting in a pattern of insulated conductive sections spaced by a non-conductive member 20, essentially forming three interlaced inductors. The catheter 31 shown in FIG. 1 utilizes this braiding in structure 8 to electrically connect the electrodes 1-4 to the proximal end of the catheter. Distal to electrode 4, the remaining wires to connect electrodes 1-3 to the proximal end can be continued as an insulated wire bundle 7, similar to the wire bundle D shown in FIG. I or as braiding coils successively reduced by one member as electrical connections are made to the subsequent distal electrodes. The lead 33 in FIG. 3 utilizes this braiding in structures 11 and 12, a coaxial arrangement, to connect the electrodes 1 and 2 to the proximal end of the lead. The embodiment here takes advantage of the multi filar nature to give a redundant connection to the electrodes. Another embodiment utilizes the multiple insulated conductive pathways to create a co-radial structure connecting the electrodes to the proximal end of the lead. It will be understood by those of skill in the art that more or fewer wires 24 and non-conductive members 20 can be used in varying quantities in the set 38 of FIG. 11, resulting in a variety of patterns exhibiting varying electrical characteristics while maintaining similar mechanical behavior.

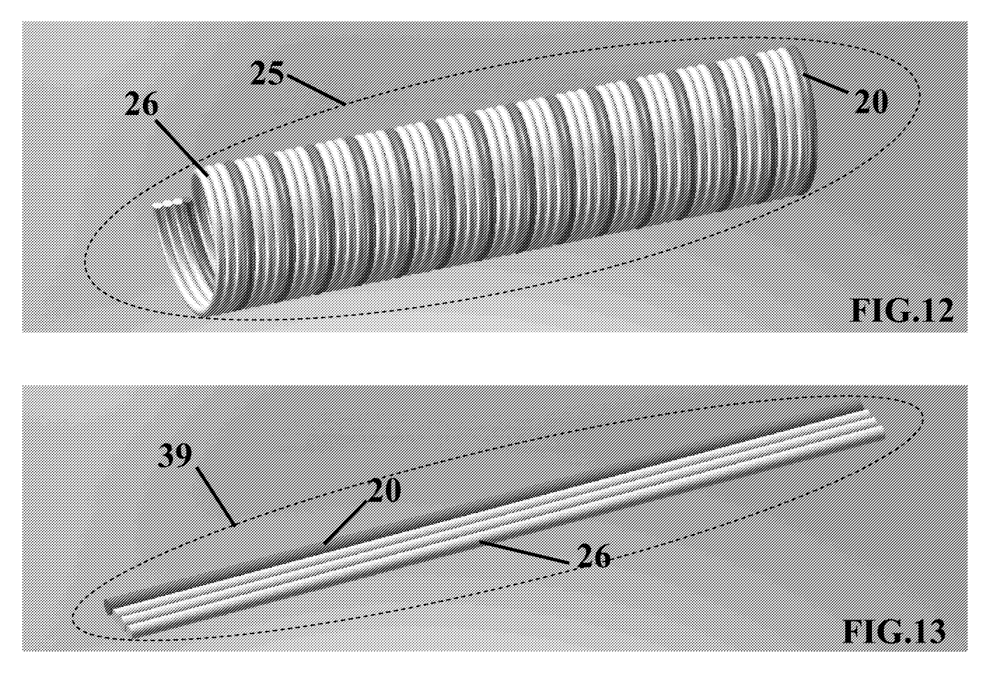

Referring to FIG. 12, braiding coil 25 includes four wires (three fully non-insulated wires 26 and one non-conductive member 20) coiled together in a quadruple helix, resulting in a pattern of conductive sections spaced by a non-conductive member 20, essentially forming a continuous inductor with pitch determined by the non-conductive member 20. A possible embodiment includes lead 33 of FIG. 3 where the structures 11 and 12 utilize braiding coil 25 of FIG. 12 instead of coil 23 of FIG. 10. It will be understood by those of skill in the art that more or fewer wires 26 and non-conductive members 20 can be used in the set 39 of FIG. 13 in varying quantities, resulting in a variety of patterns exhibiting varying electrical characteristics while maintaining similar mechanical behavior.

It will be apparent to those of skill in the art that wires of substantially equal or differing lengths and/or conductivities can be employed within multi-wire structures such as described herein. It will also be apparent to those of skill in the art that wires of different cross-section, including size and geometry (circular, square, rectangular, etc.) can be employed within multi-wire structures such as described herein.

By varying the winding patterns of the braiding coils used in the medical device, well-defined low pass or band stop filter sections of the coil can be created to reduce or eliminate alternating currents at or above specific target frequencies. Using insulated and non-insulated coil sections, localized inductors in the conductive pathway can be formed. Further, self-resonance frequencies of individual inductor sections can be adjusted using a multi-wire structure (double, triple, quadruple, etc. helix) incorporating conductive, nonconductive, and/or low conductive wire and/or sections of wire. The self-resonance of the inductor sections of the coil can be adjusted to coincide with the highest operating frequency desired. The coil pattern can be adjusted such that a variety of inductor sections with different self-resonant frequencies are formed. These sections form a string of “tank circuits” at various frequencies, which thereby block currents at specific desired frequencies.

It will be apparent to those of skill in the art that the principles above can be equally applied to both the outer and inner wire coils, that is, a co-axial structure as for example, shown in FIGS. 1, 2 and 3. It will also be apparent to those of skill in the art that the inner and outer wire coil structures can utilize different embodiments of the invention as shown in FIG. 1, or similar coil patterns, as shown in FIGS. 2 and 3.

It will be apparent to those of skill in the art that the principles described above can be applied to co-radial structures, utilizing a mix of embodiments presented above.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

What is claimed is:

1. A medical device comprising:
an elongated body; and
an electrically conductive coil wrapped around the elongated body and covering at least a lengthwise portion of the body, the coil including a pattern of insulated sections and non-insulated sections.

2. The device of claim 1, wherein the coil comprises a single, continuous wire.

3. The device of claim 2, wherein the pattern comprises alternating insulated sections and non-insulated sections.

4. The device of claim 3, wherein the insulated sections and the non-insulated sections are of substantially equal length.

5. The device of claim 4, wherein the length of the insulated wire sections is such as to form partial, one, or multiple insulated coil turns.

6. The device of claim 3, wherein the insulated sections and the non-insulated sections are of unequal length.

7. The device of claim 6, wherein the length of the insulated wire sections is such as to form partial, single, or multiple insulated coil turns.

8. The device of claim 3, wherein the alternating insulated sections and non-insulated sections are created by a removal process that removes partial sections from a fully insulated wire by chemical, mechanical, optical, or thermal means.

9. The device of claim 3, wherein the alternating insulated sections and non-insulated sections are created by a covering process that covers sections of a fully non-insulated wire with insulation material.

10. The device of claim 3, wherein the alternating insulated sections and non-insulated sections are created from a plurality of fully insulated and non-insulated sections.

11. The device of claim 1, wherein the coil comprises multiple wires wrapped in a helix.

12. The device of claim 11, wherein the multiple wires have different conductivities.

13. The device of claim 11, wherein at least one of the multiple wires includes alternating insulated and non-insulated sections.

14. The device of claim 13, wherein at least one of the multiple wires is an insulator.

15. The device of claim 11, wherein at least one of the multiple wires includes a fully insulated wire.

16. The device of claim 15, wherein at least one of the multiple wires is an insulator.

17. The device of claim 11, wherein at least one of the multiple wires includes a bare, non-insulated wire.

18. The device of claim 17, wherein at least one of the multiple wires includes an insulator.

19. The device of claim 1, wherein the medical device comprises a catheter for magnetic resonance imaging (MRI).

20. A medical device comprising:
an elongated body;
a first electrically conductive coil wrapped around the elongated body and covering at least a lengthwise portion of the body, the first coil including a pattern of insulated sections and non-insulated sections; and
a second electrically conductive coil wrapped around the elongated body and covering at least the lengthwise portion of the body, the second coil including a pattern of insulated sections and non-insulated sections,
wherein the first and second coils are arranged in a coaxial relationship relative to the elongated body.
